(19) United States
(12) Patent Application Publication
(10) Pub. No.: US 2018/0070956 A1
(43) Pub. Date: Mar. 15, 2018

(54) METHOD OF MANUFACTURING AN INFLATABLE COMPRESSION DEVICE

(71) Applicant: Merit Medical Systems, Inc., South Jordan, UT (US)

(72) Inventors: Fred Lampropoulos, Salt Lake City, UT (US); Justin Lampropoulos, Lehi, UT (US); Tyler Rees, Draper, UT (US)

(21) Appl. No.: 15/705,759

(22) Filed: Sep. 15, 2017

Related U.S. Application Data
(60) Provisional application No. 62/395,080, filed on Sep. 15, 2016.

(51) Int. Cl. A61B 17/132; A61B 17/135; B41J 2/21

(52) U.S. Cl. CPC A61B 17/1322; A61B 2017/00526; B41J 2/21; A61B 17/135

(57) ABSTRACT

A method of manufacturing an artery compression device. The method includes obtaining a polymer substrate with a first surface and a second surface and applying a colorant layer to the first surface of the polymer substrate. The polymer substrate is coupled to a fastener. The polymer substrate is also coupled to a semi-rigid member, the semi-rigid member including an outer surface and an inner surface and a flexible member to the semi-rigid member. An inflatable chamber is at least partially defined by the flexible member.

### RELATED APPLICATIONS

This application claims priority to U.S. Provisional Application No. 62/395,080 filed on Sep. 15, 2016 and titled “Method of Manufacturing an Inflatable Compression Device” which is hereby incorporated by reference in its entirety.

### TECHNICAL FIELD

The present disclosure relates generally to the field of medical devices. More particularly, some embodiments relate to manufacturing compression devices, including radial artery compression devices with an inflatable chamber.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 illustrates a top view of a compression device according to some embodiments.

FIG. 2 illustrates an exploded view of the compression device of FIG. 1.

FIG. 3 illustrates a perspective view of the compression device of FIG. 1.

FIG. 4 illustrates a cross-section view of the compression device of FIG. 1 taken along the line 4-4.

FIG. 5 illustrates a perspective view of a compression device according to some embodiments.

FIG. 6 illustrates a side view of the compression device of FIG. 5.

FIG. 7 illustrates a perspective bottom view of the compression device of FIG. 5.

### DETAILED DESCRIPTION

Numerous medical procedures involve insertion of one or more elongate medical devices into the vasculature of a patient. Some of these interventional procedures involve delivery of a medical device through a radial artery of the patient. Achieving hemostasis during and/or after an interventional procedure that involves puncturing the radial artery may present certain challenges.

To facilitate hemostasis at the radial access site, pressure may be applied slightly upstream of the skin puncture site. Such pressure may prevent or reduce the leakage of blood from the arteriotomy site and promote hemostasis. Certain embodiments described herein facilitate the application of pressure to promote hemostasis at a radial access site.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities. Thus, two components may be coupled to each other even though they are not in direct contact with each other. For example, two components may be coupled to one another through an intermediate component. The phrase “attached to” refers to interactions between two or more entities which are in direct contact with each other and/or are separated from each other only by a fastener of any suitable variety (e.g., an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a radial artery compression device or a component thereof is the end that is furthest from the attachment point of the arm of the patient during ordinary use of the device. The proximal end refers to the opposite end, or the end nearest the patient during ordinary use. When used as a directional term, the term “radial” refers to the direction pointing from the center of the arm or hand to the thumb-side portion of the arm or hand. The term “ulnar” refers to the opposite direction. The particular volumes recited herein refer to the volumes of fluid that are delivered from a syringe that holds the recited amount of fluid at atmospheric pressure. For example, an inflatable chamber has a capacity of 15 mL if it is capable of receiving 15 mL of air from a syringe that holds 15 mL of air at atmospheric pressure.

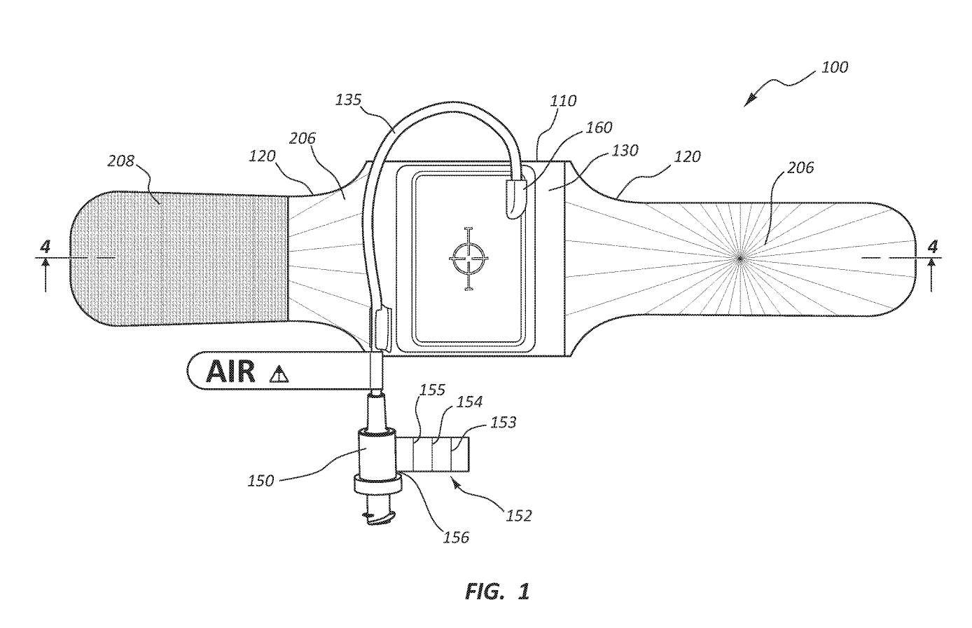

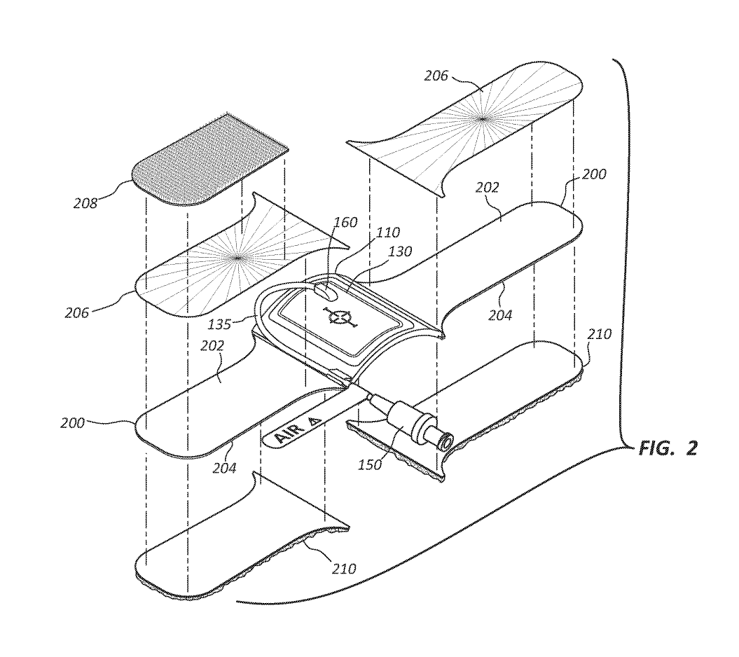

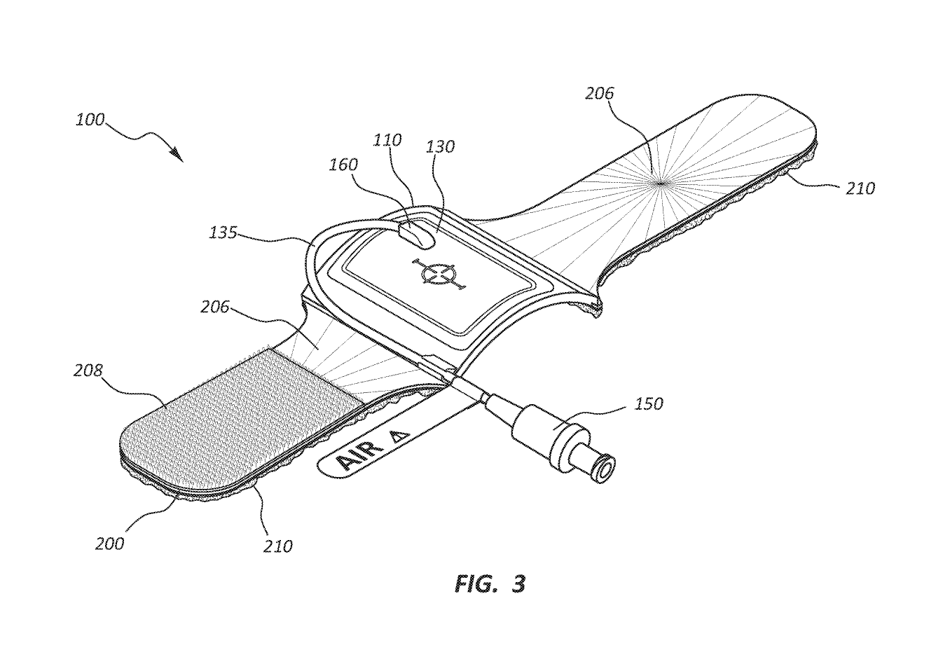

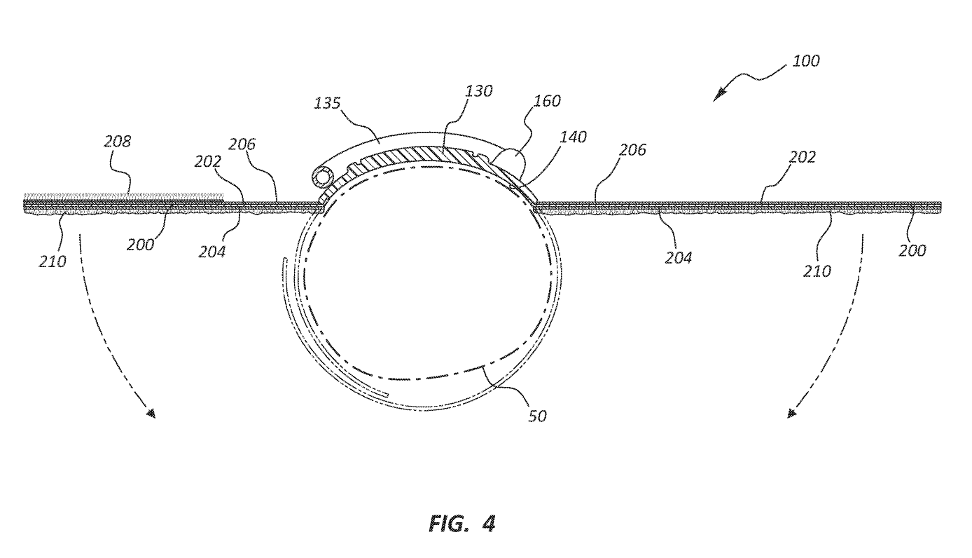

FIGS. 1-4 provide alternative views of a compression device 100 for compressing a portion of the vasculature, such as the radial artery. As shown in FIGS. 1-4, the radial artery compression device 100 may include an inflatable portion 110 and a wristband 120. FIG. 1 illustrates a top view of a compression device 100. FIG. 2 illustrates an exploded view of the compression device 100 to illustrate the various components. FIG. 3 illustrates a perspective view of the compression device 100 and FIG. 4 illustrates a cross-section view through the line 4-4 in FIG. 1 to show the various layers of the compression device 100.

The inflatable portion 110 includes a semi or substantially rigid member, such as frame 130 and a flexible sheet 140 which may be inflated to compress the radial artery.

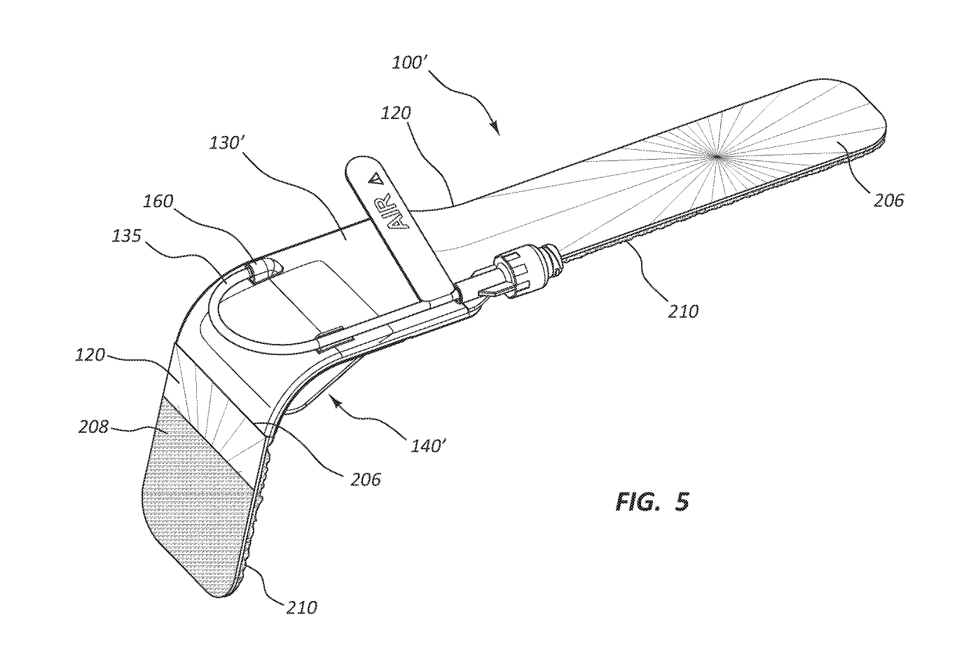

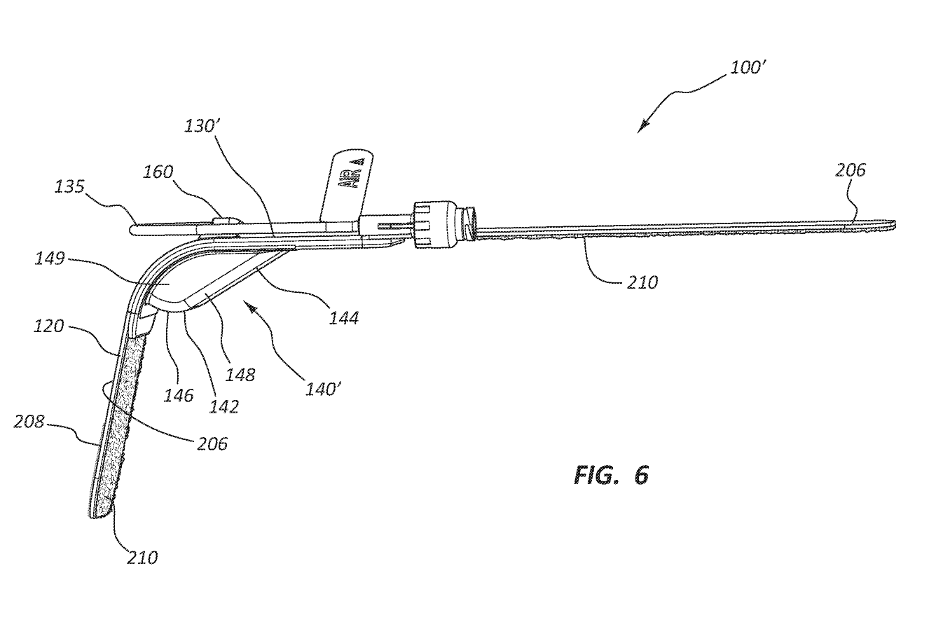

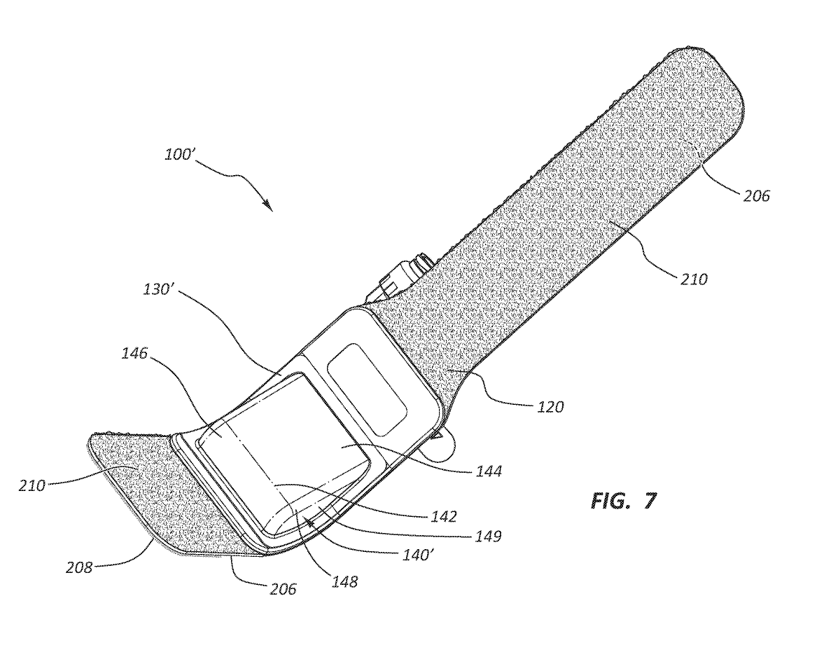

The substantially rigid frame 130 may include an outer surface and an inner surface. In some embodiments, the substantially rigid frame 130 is contoured to curve around a thumb-side portion and/or a little finger side portion of the wrist of the patient 50. In the embodiment shown in FIGS. 1-4, the frame 130 is shaped as a curved (e.g., arched) sheet. The outer surface of the frame 130 (or a portion thereof) may be convex, while the inner surface of the frame 130 (or a portion thereof) may be concave. In some embodiments, as shown in FIGS. 5-7, compression device 100′ includes a substantially rigid frame 130′ with a substantially straight section configured to be disposed adjacent an underside (i.e., a palmar side) of a wrist of the patient 50. In some embodiments, the substantially rigid frame 130 (or a portion thereof) is transparent. The substantially rigid frame 130 is more rigid than the wristband 120 (including the polymer substrate 200 discussed below) and the flexible sheet 140.

As illustrated in FIGS. 1-4, the flexible sheet 140 may be coupled to the frame 130. For example, in some embodiments, the flexible sheet 140 includes a peripheral portion that is attached to the frame 130 and a central portion that is not attached to the frame 130. In some embodiments, the peripheral portion of the flexible sheet 140 is attached to the frame 130 via welding or an adhesive. The flexible sheet 140 may be made from any suitable material, such as polyurethane or polyvinyl chloride (PVC). In some embodiments, the material of the flexible sheet is stretchable. In the depicted embodiment in FIGS. 1-4, the flexible sheet is substantially rectangular in shape, although other shapes are also within the scope of this disclosure. In some embodiments, the flexible sheet 140 (or a portion thereof) is transparent. For example, in some embodiments, both the substantially rigid frame 130 (or a portion thereof) and the flexible sheet 140 (or a portion thereof) are transparent, thereby allowing a practitioner to view a radial access site through the frame 130 and the flexible sheet 140. In some embodiments, the practitioner may need to view through only two layers (e.g., the frame 130 and the flexible sheet 140) to view the radial access site. Viewing through only two layers may provide improved visual clarity relative to embodiments in which the radial access site is viewed through more than two layers or parts. The substantially rigid frame 130 and the flexible sheet 140 may form the inflatable chamber. For example, the inner surface of the frame 130 and the flexible sheet 140 may at least partially define the inflatable chamber 126. Stated differently, a wall of the inflatable chamber may be defined by the frame 130. In this fashion, the inflatable chamber may be defined by both a first portion (e.g., the substantially rigid frame 130) of the radial artery compression device 100 that does not change size or shape as the inflatable chamber is inflated and a second portion (e.g., the flexible sheet 140) of the radial artery compression device 100 that does change in size or shape as the inflatable chamber is inflated.

In other embodiments, as illustrated in FIGS. 5-7, compression device 100′ may have a thermoformed balloon 140′. The thermoformed balloon 140′ may be non-stretchable. The shape of the thermoformed balloon 140′ is predetermined and may add compression to a certain area of the patient's wrist 50. For example, the thermoformed balloon 140′ may compress the radial artery. The thermoformed balloon 140′ illustrated in FIG. 5-7 has an outer surface with a straight portion 144 and a curved (convex) portion 146. A majority of the thermoformed balloon 140′ is located within the concave portion of the frame 130′. The thermoformed balloon 140′ may include a ridge or ridges 142 that further define the shape of the thermoformed balloon 140′. In the illustrated embodiment, ridge 142 is the portion of the thermoformed balloon 140′ that transitions from the straight portion 144 to the curved portion 146. The thermoformed balloon 140′ may further include a curved transition 148 from the straight portion 144 and curved portion 146 to a side wall 149. In some embodiments, the radial artery compression device 100 includes tubing 135 that extends from a first aperture in the substantially rigid frame 130 to a valve 150. The tubing 135 and the valve 150 may be in fluid communication with the inflatable chamber that is formed by the substantially rigid frame 130 and the flexible sheet 140 or thermoformed balloon 140′. In some embodiments, the valve 150 is configured to allow fluid (e.g., air, etc.) to flow through the valve 150 when the valve 150 is coupled to an inflation device (e.g., a syringe), but prevents fluid flow through the valve 150 when the valve 150 is not coupled (i.e., detached from) the inflation device. In other words, the valve 150 may maintain a positive fluid pressure within the inflatable chamber after the inflation device has been uncoupled from the valve 150. In some embodiments, valve 150 may have an adaptive configuration where the valve 150 has a cap with a hole and the syringe has a mated connector. This adaptive configuration may be configured to prevent a user from connecting a standard syringe to the valve 150. In other words, the system may be configured with a unique connection between the valve 150 and an inflation source. This may also prevent or minimize instances of coupling the inflation source to other medical tubing (such as a catheter disposed within a patient's body) by mistake. In the depicted embodiment, the tubing 135 is coupled to the frame 130 via a connector 160 that protrudes from the outer surface of the frame 130.

In some embodiments, as illustrated in FIG. 1, the valve 150 may include a pressure gauge 152 to indicate to a medical professional the pressure within inflatable device 110. Pressure gauge 152 may enable the medical professional to obtain a predetermined pressure to perform the procedure.

Pressure gauge 152 may include a plurality of indicia, such as 153, 154, and 155 to indicate the pressure within inflatable device 110. Indicia may be in pressure units (e.g., psi mmHg, bar, atm, Kpa, etc.) or may be color coated. For example, 153 may be green to indicate a safe pressure, 154 may be yellow to indicate a warning pressure, and 155 may be red to indicate high pressure. Pressure gauge 152 may comprise any variety of indicia and may be analog or digital. Pressure gauge 152 may further include a release valve 156 which releases fluid if inflatable device 110 reaches a predetermined high pressure. Release valve 156 may help ensure that excess pressure to not applied to the radial artery.

Alternatively, various instruments may be used in place of or in addition to pressure gauge 152. For example, an instrument coupled to the system may indicate the amount of fluid inputted into inflation device 110 instead of the pressure in inflation device 110. Also pressure gauge 152 may be on balloon, inflation source, and so on.

The wristband 120 may be coupled to the frame 130. For example, the wristband 120 may include a first strap that is coupled to one side of the frame 130 and a second strap that is coupled to an opposite side of the frame 130. The wristband 120 may be configured to secure the frame 130 adjacent to the wrist of the patient 50. In some embodiments, the entire wristband 120 (or a portion thereof) is opaque. In some embodiments, the wristband 120 is colored and/or decorated, as discussed in more detail below.

During manufacture of the arterial compression device 100, a polymer substrate 200 is obtained as a base for the wristband 120. The polymer substrate 200 may be a polyvinyl chloride (PVC) laminate. In other embodiments the polymer substrate 200 may be a polyurethane laminate. The polymer substrate 200 has a thickness between 0.001 and 0.025 inches. The polymer substrate 200 includes a first surface 202 and a second surface 204. During manufacture, a colorant layer 206 is applied to the first surface 202 of the polymer substrate 200, as will be discussed in more detail below. The polymer substrate is also coupled to a fastener, as also described in more detail below. The wristband 120 with the colorant layer 206 is coupled to the inflatable member 110. In some embodiments, the wristband 120 is coupled to the substantially rigid frame 130. In some embodiments, the wristband 120 couples to the substantially rigid frame 130 by an ultraviolet curable adhesive. This may be done by attaching the nylon loops 208 to the substantially rigid frame 130 using the UV curable adhesive However, other known attachment methods, such as radio frequency (RF) welding, may be used as would be appreciated by one skilled in the art with the benefit of this disclosure. For example, the polymer substrate 200 may be RF welded to the substantially rigid frame 130. In some embodiments, the inflatable device 110 couples to the wristband 120 through a pocket in the wristband 120 configured to receive the frame 130 and the flexible sheet 140. In these embodiments, the polymer substrate extends along the entire compression device 100, including the frame 130.

In some embodiments, as shown in FIGS. 1-4, the fastener may be a hook and loop fastener (e.g., Velcro). In some embodiments, nylon loops 210 are coupled to the second surface 204 of the polymer substrate 200. The nylon loops 210 may cover the entire second surface 204 of the polymer substrate 200. In some embodiments, the polymer substrate 200 is obtained with the nylon loops 210 coupled to the second surface 204. In such embodiments, the polymer substrate 200 obtained with the nylon loops has a thickness between 0.020 to 0.085 inches. Corresponding hooks 208 are coupled to the first surface 202, optionally via a portion of the colorant layer 206 there between. The hooks 208 may be coupled by RF welding in some embodiments. However, other known attachment methods may be used as would be appreciated by one skilled in the art with the benefit of this disclosure. For example, hook 208 may be coupled by adhesive. When attached to a user's limb, a portion of the nylon loops 210 on the second surface 204 of the polymer substrate fasten to at least a portion of the hooks 208 on the first surface 202 (e.g., the portion of the colorant layer 206).

Although a hook 208 and loop 210 fastener is shown in FIGS. 1-4, any type of known fastener may be used to secure the radial artery compression device 100 to the arm of the patient 50, as would be readily appreciated by one skilled in the art with the benefit of this disclosure. For example, the fastener may be an adhesive applied to a portion of the second surface 204 to attach to the colorant layer 206, or alternatively, a portion of the first surface 202, when wrapped around a patient's limb. In other embodiments, the fastener may comprise a buckle or a button.

As noted above, a colorant layer 206 is applied to the first surface 202 of the polymer substrate 200. In some embodiments, a band design choice is obtained from a purchaser prior to the colorant layer 206 being applied. This can be selected by the purchaser manually or through an automated system, such as an application on a tablet, computer, or smartphone device. The colorant layer 206 is then applied consistent with the band design choice. In some embodiments, the colorant layer 206 may include multiple colors and/or be a pattern. In some embodiments, the color of the colorant layer 206 may be chosen based on a color coded for radial artery access. For example, other colors may be used for femoral compression or to distinguish from other medical bandages or devices.

The colorant layer 206 may be applied by printing directly onto the first surface 202 of the polymer substrate 200. For example, the printing may be done by inkjet printing, screen printing, or pad printing. In some embodiments, to print directly onto the first surface 202 of the polymer substrate 200, the polymer substrate 200, including the nylon loops 210 in some embodiments, is flattened. In some embodiments, the ink is an ultraviolet (UV) curable ink. The UV curable ink is applied to the first surface 202 of the polymer substrate 200 and then cured by the printer. In some embodiments, multiple layers of ink are printed onto the first surface 202 of the polymer substrate to correspond to a desired resolution. For example, applying the colorant layer 206 may include applying two layers of colorant to the first surface 202 of the polymer substrate 200. The thickness of the colorant layer 206 may be approximately 0.00025 to 0.005 inches, such as approximately 0.001 inches to 0.002 inches. In some alternative embodiments, the colorant layer 206 may first be printed onto a material with a bottom adhesive layer. The colorant layer 206 is then coupled to the first surface 202 of the polymer substrate 200.

In some embodiments, a primer may be applied to the first surface 202 before applying the colorant layer 206. The primer may be, for example, an acrylic ester, an aliphatic monomer with an acrylic oligomer, or an alcohol. Some embodiments may also include applying a clear coat to the colorant layer 206 after the colorant layer is applied to the first surface 202.

After the colorant layer 206 is applied to the first surface 202, the polymer substrate 200, the nylon loops 210, and the colorant layer 206 may be die cut into wristband 120. Wristband 120 is then coupled to the inflatable device 110. The hooks 208 may be coupled before or after the die cut.

Although a radial compression device is shown in FIGS. 1-4, the compression device 100 may also be used as a femoral compression device, as will be appreciated by one having ordinary skill in the art with the benefit of this disclosure. A femoral compression device would be manufactured in an equivalent manner as the described radial compression device 100.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A method of manufacturing an artery compression device, the method comprising:
obtaining a polymer substrate with a first surface and a second surface;
applying a colorant layer to the first surface of the polymer substrate;
coupling the polymer substrate to a fastener;
coupling the polymer substrate to a semi-rigid member, the semi-rigid member including an outer surface and an inner surface; and
coupling a flexible member to the semi-rigid member, wherein an inflatable chamber is at least partially defined by the flexible member.

2. The method of claim 1, wherein the inflatable chamber is at least partially defined by the inner surface of the semi-rigid member.

3. The method of claim 1, wherein the polymer substrate is a polyvinyl chloride (PVC) laminate or a polyurethane laminate.

4. The method of claim 3, wherein the polymer substrate is opaque.

5. The method of claim 1, wherein the semi-rigid member is more rigid than the polymer substrate and the flexible member.

6. The method of claim 1, wherein the flexible member is a thermoformed balloon.

7. The method of claim 1, wherein a flexible tubing extends from a first aperture in the semi-rigid member to a valve.

8. The method of claim 7, wherein the valve further comprises a pressure gauge.

9. The method of claim 1, wherein the second surface of the polymer substrate includes nylon loops, and coupling the polymer substrate to a fastener includes coupling a hook material corresponding to the nylon loops to at least a portion of the first surface of the polymer substrate.

10. The method of claim 1, wherein the polymer substrate has a thickness between 0.001 and 0.025 inches.

11. The method of claim 1, wherein applying the colorant layer includes printing the colorant layer on the first surface of the polymer substrate, the printing including at least one of the following: inkjet printing, screen printing, and pad printing.

12. The method of claim 1, wherein applying the colorant layer includes printing a pattern using an inkjet printer and ultraviolet curable ink onto the first surface of the polymer substrate and curing the ultraviolet curable ink.

13. The method of claim 1, wherein the colorant layer is a material with a color pre-printed on a first side and an adhesive material on a second side, wherein the adhesive material attaches to the first surface of the polymer substrate.

14. The method of claim 1, further comprising applying a primer to the first surface of the polymer substrate prior to applying the colorant layer.

15. The method of claim 1, further comprising applying a clear coat layer to the colorant layer after applying the colorant layer to the first surface of the polymer substrate.

16. The method of claim 1, further comprising cutting the polymer substrate to a wrist band shape after applying the colorant layer.

17. A method of manufacturing an artery compression device, the method comprising:
providing a polymer substrate;
applying a colorant layer to the polymer substrate;
coupling the polymer substrate to a fastener;
coupling the polymer substrate to a transparent inflatable member with an inflatable chamber at least partially defined by a semi-rigid member and a flexible member.

18. The method of claim 17, wherein applying the colorant layer includes applying two colorant layers.

19. The method of claim 17, further comprising:
obtaining a band design choice from a purchaser; and
applying the colorant layer consistent with the band design choice to the first surface of the polymer substrate.

20. A method of manufacturing a compression device, the method comprising:
obtaining a band design choice from a purchaser;
obtaining a polymer substrate with a first surface and a second surface;
applying a colorant layer consistent with the band design choice to the first surface of the polymer substrate;
coupling the polymer substrate to a fastener;
coupling the polymer substrate to an inflatable member with an inflatable chamber at least partially defined by a semi-rigid member and a flexible member.
